(19) United States
(12) Patent Application Publication
(10) Pub. No.: US 2020/0395102 A1
(43) Pub. Date: Dec. 17, 2020

(54) POLYMER PHYSICAL PROPERTY PREDICTION DEVICE, RECORDING MEDIUM, AND POLYMER PHYSICAL PROPERTY PREDICTION METHOD

(71) Applicant: SHOWA DENKO K.K., Tokyo (JP)

(72) Inventors: Takuya MINAMI, Tokyo (JP); Yoshishige OKUNO, Tokyo (JP)

(21) Appl. No.: 17/008,999

(22) Filed: Sep. 1, 2020

Related U.S. Application Data
(63) Continuation-in-part of application No. PCT/JP2019/008704, filed on Mar. 5, 2019.

(30) Foreign Application Priority Data
Mar. 9, 2018 (JP) 2018-043171

(51) Int. Cl. G16C 20/30; C08F 110/02; G01N 33/44

(52) U.S. Cl. CPC G16C 20/30; G01N 33/442; C08F 110/02

(57) ABSTRACT

A polymer physical property prediction device includes a processor, and a memory storing program instructions that cause the processor to read a structural unit from a storage unit and use the structural unit to calculate numbers each indicating how many substructures are in a polymer, the polymer being formed of repetition of the structural unit, calculate a number indicating how many atoms are in the structural unit, calculate number densities of the substructures from the numbers of substructures and the number of atoms in the structural unit, construct a regression model that predicts a physical property value by using an experimental value of a physical property of the polymer and the number densities of the substructures, input a polymer structure of which the physical property value is to be predicted, and predict the physical property value corresponding to the input polymer structure by using the regression model.

### CROSS-REFERENCE TO RELATED APPLICATIONS

This application is a continuation-in-part application of International Application No. PCT/JP2019/008704 filed on Mar. 5, 2019, which is based upon and claims priority to Japanese Priority Application No. 2018-043171 filed on Mar. 9, 2018, the entire contents of which are hereby incorporated by reference.

### BACKGROUND OF THE INVENTION

### 1. Field of the Invention

The present invention relates to a device that predicts the physical properties of a polymer (hereinafter “polymer physical property prediction device”, a recording medium, and a polymer physical property prediction method.

### 2. Description of the Related Art

In recent years, machine learning has been used to predict physical properties of organic compounds such as polymers by using a computer.

Conventional techniques for predicting. physical properties of polymers use various structure descriptors configured by humans to predict physical properties. Techniques for mechanically identifying substructure patterns of molecules and calculating structure descriptors based on algorithms have also been proposed.

Conventionally, it is necessary for a human to determine a structural pattern, such as the number of carbon atoms, that serves as a structure descriptor of a polymer in advance. With respect to this, there is a method for automatically extracting substructures of low-molecular weight organic molecules, but there is a problem that the accuracy of predicting the physical properties of a polymer is low. Thus, the method has not been applied to polymer structures.

### RELATED-ART DOCUMENTS

### Patent Documents

[Patent Document 1] Japanese National Publication of International Patent Application No. 2004-514902

### Non-Patent Document

[Non-Patent Document 1] David Rogers et al., “Extended-Connectivity Fingerprints”, American Chemical Society, J. Chem. Info. Model., Vol. 50, No. 5, 2010, pp. 742-754.

### SUMMARY OF THE INVENTION

According to one aspect, a polymer physical property prediction device includes a processor, and a memory storing program instructions that cause the processor to read a structural unit from a storage unit and use the structural unit to calculate numbers each indicating how many substructures are in a polymer, the polymer being formed of repetition of the structural unit, calculate a number indicating how many atoms are in the structural unit, calculate number densities of the substructures from the numbers of substructures and the number of atoms in the structural unit, construct a regression model that predicts a physical property value by using an experimental value of a physical property of the polymer and the number densities of the substructures, the experimental value being obtained from the storage unit, input a polymer structure of which the physical property value is to be predicted, and predict the physical property value corresponding to the input polymer structure by using the regression model.

A means to solve the above-described problem can be a program or a method for predicting the physical properties of a polymer.

According to at least one embodiment of the present invention, the physical properties of a polymer can be accurately predicted.

### BRIEF DESCRIPTION OF THE DRAWINGS

FIG. 1 is a drawing illustrating an example of a system configuration according to the present embodiment;

FIG. 2 is a drawing illustrating an example of a hardware configuration of a physical property prediction device;

FIG. 3 is a drawing illustrating a first functional configuration example of the physical property prediction device;

FIG. 4 is a drawing illustrating an example of a data configuration of a polymer structure and physical property DB;

FIG. 5 is a drawing for describing an overview of a prediction process according to the present embodiment;

FIG. 6 is a drawing illustrating an example of the number density;

FIG. 7 is a drawing for describing a difference caused when replication of a repeating unit is performed and no replication of a repeating unit is performed;

FIG. 8A is a flowchart for describing the prediction process;

FIG. 8B is a flowchart for describing the prediction process;

FIG. 9 is a drawing illustrating an example of a data configuration of a regression model management table;

FIG. 10 is a drawing illustrating a second functional configuration example of the physical property prediction device;

FIG. 11 is a drawing illustrating a third functional configuration example of the physical property prediction device;

FIG. 12 is a drawing illustrating a fourth functional configuration example of the physical property prediction device;

FIG. 13 is a drawing illustrating a functional configuration example of a physical property prediction device based on an existing technique;

FIG. 14 is a drawing illustrating an example of evaluation results;

FIG. 15 is a drawing illustrating an example of evaluation results; and

FIG. 16 is a drawing illustrating an example of a manufacturing method for manufacturing a polymer.

### DESCRIPTION OF THE EMBODIMENTS

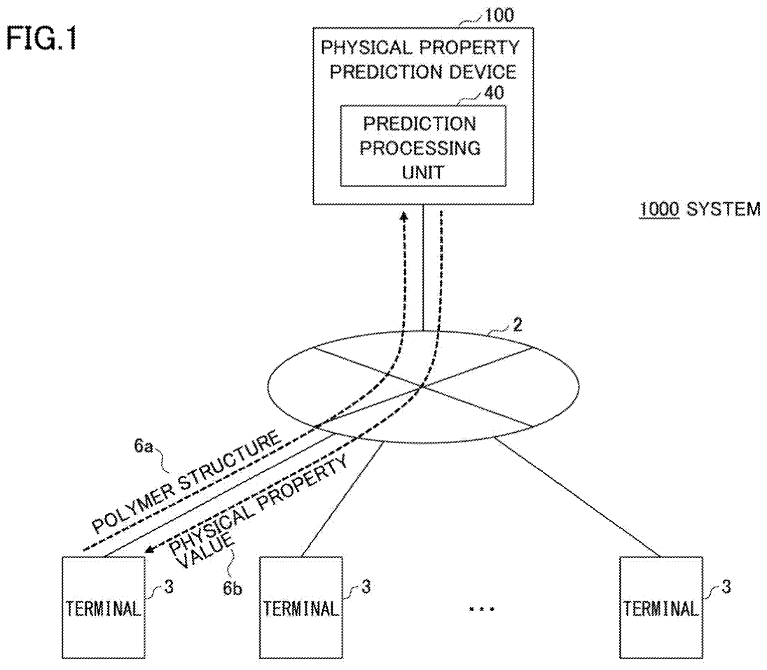

In the following, an embodiment of the present invention will be described based on the drawings. First, an example of a system configuration according to the present embodiment will be described. FIG. 1 is a drawing illustrating the example of the system configuration according to the present embodiment In FIG. 1, a system 1000 includes a physical property prediction device 100 and multiple terminals 3, and each of the terminals can be connected through a network 2.

The terminal 3 is an information processing terminal used by a user, is connected to the physical property prediction device 100, and utilizes processing performed by a prediction processing unit 40.

The physical property prediction device 100 is an information processing device including the prediction processing unit 40 that predicts a physical property of a polymer. In the physical property prediction device 100, the prediction processing unit 40 performs machine learning using a structure of the polymer and data indicating the physical property of the polymer that are provided in advance to generate a regression model. In response to receiving a polymer structure 6a from the terminal 3, a physical property value is predicted using the generated regression model with respect to the received polymer structure 6a, and a predicted physical property value 6b is sent to the terminal 3.

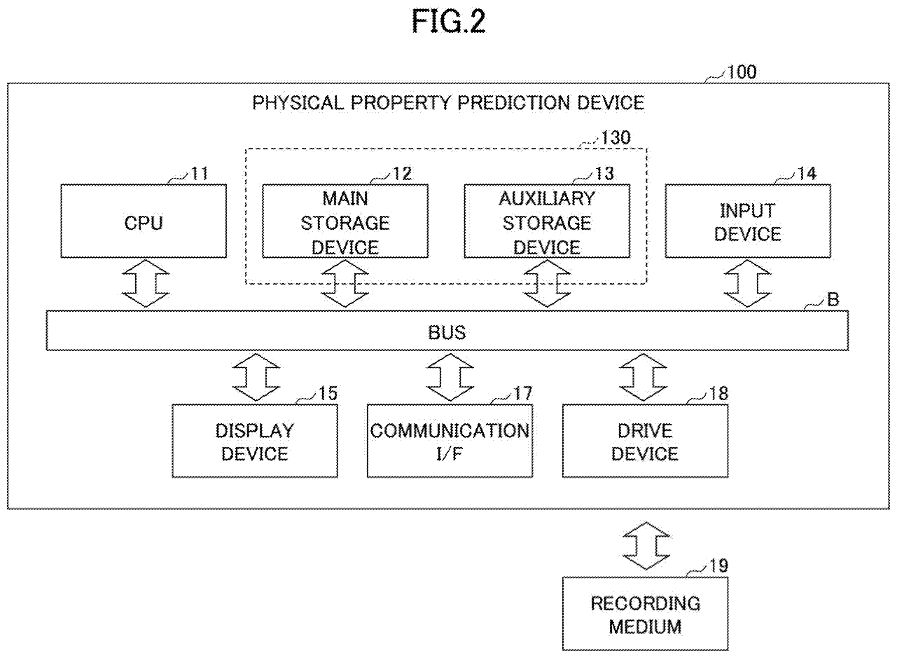

A configuration of the system 1000 is not limited to the example of FIG. 1. As an example, the physical property prediction device 100 may be utilized in a stand-alone manner. In this case, when the polymer structure 6a is input by a user operation of an input device 14 (FIG. 2), the physical property value 6b is output to a display device 15 (FIG. 2).

The physical property prediction device 100 according to the present embodiment has a hardware configuration as illustrated in FIG. 2. FIG. 2 is a drawing illustrating an example of the hardware configuration of the physical property prediction device. In FIG. 2, the physical property prediction device 100 is an information processing device controlled by a computer and includes a central processing unit (CPU) 11, a main storage device 12, an auxiliary storage device 13, the input device 14, the display device 15, a communication interface (I/F) 17, and a drive device 18, which are connected to a bus B.

The CPU 11 corresponds to a processor that controls the physical property prediction device 100 according to a program stored in the main storage device 12. As the main storage device 12, a random access memory (RAM) and a read only memory (ROM) are used to store or temporarily store a program executed by the CPU 11, data necessary for processing by the CPU 11, and data obtained by processing by the CPU 11, for example.

A hard disk drive (HDD) is used for the auxiliary storage device 13, for example, to store data such as a program for executing various processing. A portion of the program stored in the auxiliary storage device 13 is loaded into the main storage device 12 and is executed by the CPU 11 to achieve various processing. Hereinafter, the main storage device 12, the auxiliary storage device 13, and an external storage device accessible by the physical property prediction device 100 will be collectively referred to as a storage unit 130.

The input device 14 includes a mouse and a keyboard and is used by a user to input various information required for processing by the physical property prediction device 100. The display device 15 displays various necessary information under control of the CPU 11. The input device 14 and the display device 15 may be a user interface, such as an integrated touch panel. The communication I/F 17 performs communication through a network, such as a wired or wireless network. The communication by the communication I/F 17 is not limited to the wired or wireless communication. The drive device 18 interfaces a recording medium 19 set in the drive device 18 (e.g., a compact disc read-only memory (CD-ROM)) with the physical property prediction device 100.

A program for implementing the processing performed by the physical property prediction device 100 is provided to the physical property prediction device 100 through the recording medium 19, such as a CD-ROM. A program for implementing various processing according to the present embodiment, which will be described later, is stored in the recording medium 19, and the program stored in the recording medium 19 is installed in the physical property prediction device 100 through the drive device 18. The installed program can be executed by the physical property prediction device 100.

The recording medium 19 for storing the program is not limited to a CD-ROM, but may be one or more non-transitory and tangible media having a computer readable data structure. As a computer readable recording medium in addition to a CD-ROM, a portable storage medium, such as a digital versatile disk (DVD) and a USB memory, or a semiconductor memory, such as a flash memory, may be used.

[First Functional Configuration Example]

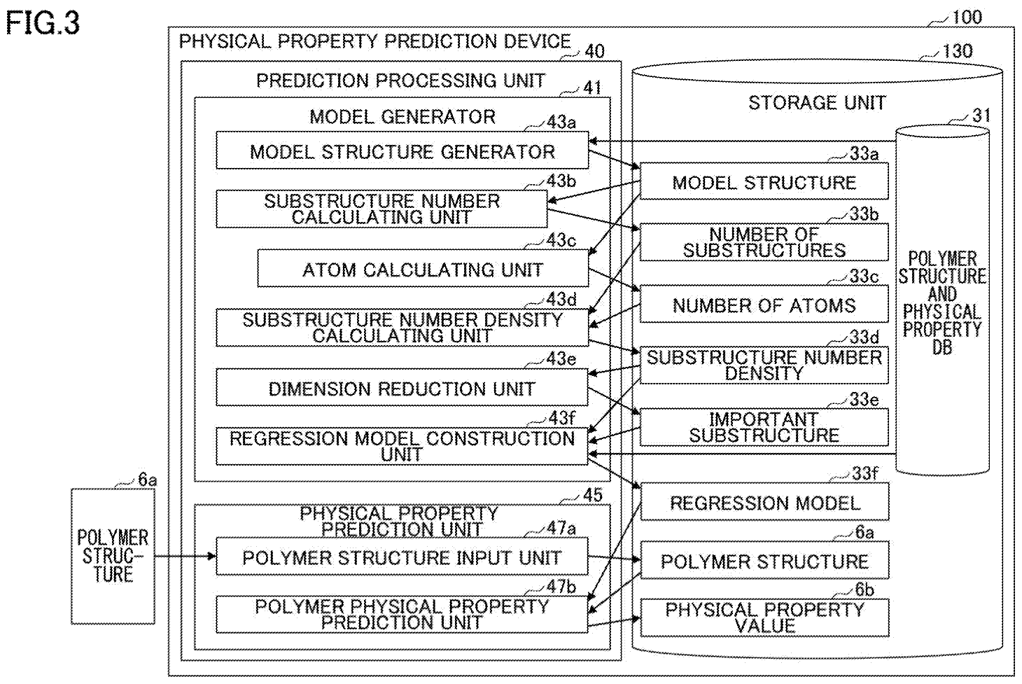

FIG. 3 is a drawing illustrating a first functional configuration example of the physical property prediction device. In FIG. 3, the physical property prediction device 100 includes the prediction processing unit 40. The prediction processing unit 40 corresponds to a prediction process performed by the CPU 11 executing a corresponding program.

The storage unit 130 stores a polymer structure and physical property DB 31, a model structure 33a, the number of substructures 33b, the number of atoms 33c, a substructure number density 33d, an important substructure 33e, a regression model 33f, a polymer structure 6a, a physical property value 6b, for example.

The prediction processing unit 40 obtains the regression model 33f for predicting the physical properties of the polymer structure 6a specified by a user by performing machine learning, predicts the physical properties of the polymer structure 6a by using the obtained regression model 33f, and outputs the obtained physical property value 6b. The prediction processing unit 40 includes a model generator 41 and a physical property prediction unit 45 as processing units.

The model generator 41 learns the physical property of the polymer structure in which a structural unit is repeatedly replicated and linked using the polymer structure and physical property DB31 and determines the regression model 33f. A structural unit forming a polymer by repetition is called a “repeating unit”. In the polymer structure and physical property DB 31, a repeating unit and an experimental value of a physical property experimentally obtained in advance are stored in association with each polymer.

The model generator 41 includes a model structure generator 43a, a substructure number calculating unit 43b, an atom calculating unit 43c, a substructure number density calculating unit 43d, a dimension reduction unit 43e, and a regression model construction unit 43f as processing units. Either or both of the model structure generator 43a and the dimension reduction unit 43e can be removed.

The model structure generator 43a obtains the repeating unit from the polymer structure and physical property DB 31 and repeats the process of replicating and linking the obtained repeating unit a predetermined number of times to generate the model structure 33a. The repeating unit includes a substructure. The repeating unit may also be an entire of a substructure. The repeating unit are linked multiple times to represent a polymer.

The generated model structure 33a is stored in the storage unit 130. The repeating unit and the model structure 33a may be represented in a predetermined form. The first functional configuration example describes the repeating unit and the model structure 33a in the simplified molecular input line entry syntax (SMILES) notation, but the notation is not limited to this.

The substructure number calculating unit 43b calculates the number of substructures 33b based on the model structure 33a. The numbers of substructures 33b are represented by an n-dimensional vector and stored in the storage 130. The atom calculating unit 43c calculates the number of atoms 33c based on the model structure 33a. The obtained number of atoms 33c is stored in the storage unit 130.

The substructure number density calculating unit 43d calculates the substructure number density 33d by using the numbers of substructures 33b and the number of atoms 33c. The substructure number density 33d can be obtained by the following equation:

substructure number density=number of substructures÷number of atoms

The obtained substructure number densities 33d are represented by a vector of the same n-dimensional vector as the vector of the substructure number 33b and are stored in the storage unit 130.

The dimension reduction unit 43e constructs a temporal regression model by using the substructure number density 33d and the experimental value, and reduces the dimensions of the temporal regression model based on a predetermined dimension reduction method to obtain the important substructure 33e. The important substructure 33e is stored in the storage unit 130. The important substructure 33e is data specifying an element (e.g., a vector) that will contribute to the prediction accuracy of the regression model 33f to be constructed in the substructure number density 33d.

For the dimension reduction method, a procedure corresponding to a regression analysis method to be used is determined in advance. For example, when the lasso regression is used, the dimensions can be reduced by removing a coefficient term of 0. When the random forest regression is used, the dimensions can be reduced by calculating the importance of explanatory variables and excluding variables that have the importance lower than or equal to a threshold. In the stepwise method, the dimensions can be reduced by searching for combinations of explanatory variables having highest prediction accuracy by adding or reducing an explanatory variable one by one when a temporal regression model is constructed.

The regression model construction unit 43f constructs the regression model 33f by performing a regression analysis based on the important substructure 33e obtained by the dimension reduction unit 43e by using the substructure number density 33d and the experimental value. The regression model 33f is stored in the storage unit 130.

In the dimension reduction unit 43e and the regression model construction unit 43f, the experimental value can be obtained from the polymer structure and physical property DB 31. The regression model 33f obtained by the regression model construction unit 43f is expressed in the following equation.

y=f(x)

By substituting the substructure number density 33d for a variable x, a predicted value y of the physical property is obtained.

To the regression analysis for obtaining the temporal regression model and the regression model 33f, any one of Lasso regression, random forest regression, a stepwise method, partial linear regression, ridge regression, kernel ridge regression, support vector regression, decision tree regression, Gaussian process regression, and so on can be applied. As an example, a temporal regression model can be obtained by the Lasso regression, and the regression model 33f can be obtained by the Gaussian process regression.

Next, the physical property prediction unit 45 predicts the physical properties of the polymer based on the polymer structure 6a by using the regression model 33f in response to an input of the polymer structure 6a specified by a user. The polymer structure 6a may specify the repeating unit by the SMILES notation or the like. The physical property prediction unit 45 includes a polymer structure input unit 47a and a polymer physical property prediction unit 47b as processing units.

The polymer structure input unit 47a inputs the polymer structure 6a specified by a user and the polymer structure 6a is stored in the storage unit 130. The polymer structure input unit 47a may input the polymer structure 6a by the communication I/F 17 receiving the polymer structure 6a through the network 2 or may input the polymer structure 6a in accordance with a user's specification by the input device 14 of the property prediction device 100.

The polymer physical property prediction unit 47b obtains the model structure, the numbers of substructures, and the number of atoms, from the polymer structure 6a, to calculate the substructure number density, and predicts the physical property value by using the regression model 33f stored in the storage unit 130. The method of obtaining the model structure, the numbers of substructures, the number of atoms, and the substructure number density based on the polymer structure 6a is similar to the method of the model generator 41.

The predicted physical property value 6b is output to the storage unit 130. When the polymer structure 6a is specified through the network 2, the obtained physical property value 6b is sent to the terminal 3 of the user and displayed. When the polymer structure 6a is specified by a user operation at the physical property prediction device 100, the obtained physical property value 6b is displayed on the display device 15.

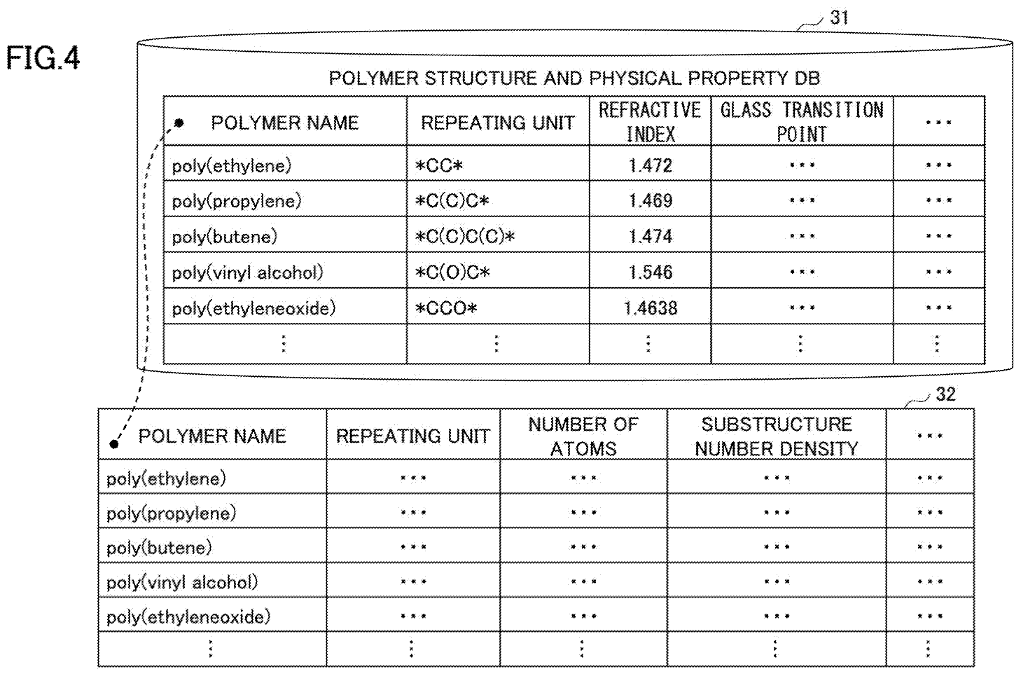

FIG. 4 is a drawing illustrating an example of a data configuration of the polymer structure and physical property DB. In FIG. 4, the polymer structure and physical property DB 31 is a database that manages the repeating unit and experimental values of various physical properties, such as a refractive index, for each polymer, and includes items such as a polymer name, the repeating unit, the refractive index, and a glass transition point.

The polymer name indicates specific information specifying the polymer structure. An example in which the repeating unit is represented by the SMILES notation is used for the following description, but the notation of the repeating unit is not limited to the SMILES notation. The refractive index, the glass transition point, and the like are examples of various physical properties of the polymer experimentally obtained in advance, and the experimental values are shown for each name of the physical property.

In this example, a repeating unit “*CC*” is associated with a polymer name “poly(ethylene)” and the experimental values, such as the refractive index “1.472”, are shown. Similarly, an experimental value of each physical property is shown for another polymer.

In the model generator 41, referenced data, generated data, and the like may be managed in a work table 32. The work table 32 is a table in which calculated values are stored for each repeating unit of the polymer of which the substructure number density 33d is obtained, and includes items such as the polymer name, the repeating unit, the number of atoms, and the substructure number density.

The polymer name indicates a polymer name registered in the polymer structure and physical property DB 31. The number of substructures indicates the number of substructures 33b obtained by the substructure number calculating unit 43b. The number of atoms indicates the number of atoms 33c obtained by the atom calculating unit 43c. The substructure number density indicates the substructure number density 33d obtained by the substructure number density calculating unit 43d.

The substructure number density of the work table 32 and the experimental values of the physical property of the polymer structure and physical property DB 31 are used for the machine learning by the regression analysis. Thus, the work table 32 may be associated with the polymer structure and physical property DB 31 by the polymer name. Here, the data configuration example of the polymer structure and physical property DB 31 and the work table 32 illustrated in FIG. 4 is only one example, but is not limited to this example.

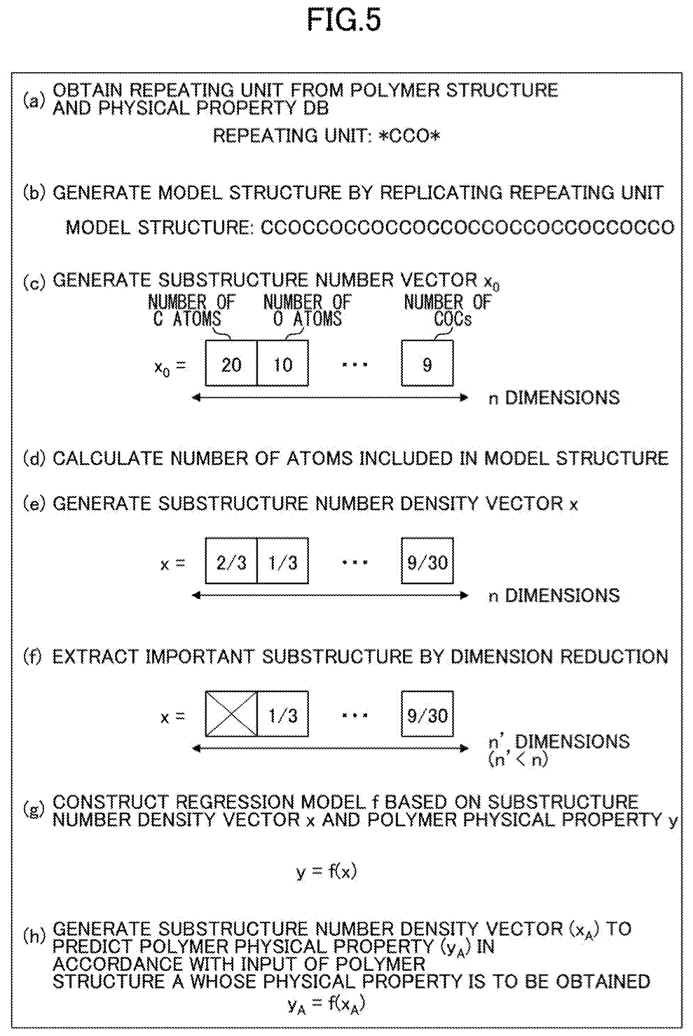

An outline of an entire prediction process performed by the prediction processing unit 40 will be described using an example of the repeating unit. FIG. 5 is a drawing for describing the overview of the prediction process according to the present embodiment. As illustrated in FIG. 5, in the model generator 41 of the prediction processing unit 40, the model structure generator 43a obtains the repeating unit from the polymer structure and physical property DB 31 (as described in (a) of FIG. 5). For example, a repeating unit “*CCO*” is obtained. In the following description, “*” indicating a start and an end of a certain structure in the SMILES notation will be omitted.

The model structure generator 43a uses the obtained substructure as a repeating unit, and replicates the repeating unit to generate the model structure 33a (as described in (b) of FIG. 5). In this example, the model structure generator 43a repeats replicating the repeating unit “CCO” to the right a predetermined number of times, to generate the model structure 33a “CCOCOCOCCCOCCCOCCCOCCCOCCCOCCO”, in which the repeating unit “CCO” are linked multiple times, in the memory unit 130.

The substructure number calculating unit 43b generates a substructure number vector x₀ (i.e., the numbers of substructures 33b) of the structure model 33a (as described in (c) in FIG. 5). The substructure number vector x₀ is represented in n dimensions by a structure descriptor, such as extended circular finger print (ECFP), atom pair, topological torsion, and neural finger print. In the first functional configuration example, a structure descriptor represented by the number of C atoms, the number of O atoms, the number of COCs, or the like is used for the description. However, the structure descriptor is not limited to this.

In the structure model “CCOCCCOCCCOCCCOCCCOCCCOCCO”, an n-dimensional substructure number vector x₀, which consists of the number of C atoms “20”, the number of O atoms “10”, . . . , and the number of COCs “9”, is generated. A structure assigned to each vector, such as “C”, “O”, “COC”, indicates the type of the substructure of the polymer. Here, n is an arbitrary natural number, and is usually about 256 to 2048.

The atom calculating unit 43c calculates the number of atoms included in the model structure 33a (as described in (d) of FIG. 5). In the structure model “CCOCOCOCOCCCOCCCOCCCOCCCO”, the number of atoms is “30”.

The substructure number density calculating unit 43d divides the substructure number vector x₀ (i.e., the numbers of substructures 33b) obtained by the substructure number calculating unit 43b in (c) of FIG. 5 by the number of atoms 33c obtained by the atom calculating unit 43c in (d) of FIG. 5 to generate the substructure number density vector x (i.e., the substructure number densities 33d) in (e) of FIG. 5.

Specifically, in the structure model “CCOCCCOCCOCCOCCCOCCCOCCCOCCCOCCO”, each value of the substructure number vector x₀ is divided by the number of atoms “30” to obtain the substructure number density vector x representing the C atom number density “⅔ (=20/30)”, the O atom number density “⅓ (=10/30)”, . . . , and the COC density “ 9/30.” The number of dimensions of the substructure number density vector x is n, which is the same number as the number of dimensions of the substructure number vector x₀.

For each record of the polymer structure and physical property DB 31, (a) of FIG. 5 to (e) of FIG. 5 are repeated. The number of substructures 33b, the number of atoms 33c, and the substructure number density 33d that are obtained may be stored in the work table 32 in association with the polymer name.

Before constructing the regression model 33f, a temporal regression model is constructed by the dimension reduction unit 43e and the dimension reduction is performed (in (f) of FIG. 5). This can reduce n dimensions to n′ dimensions (n′<n). By the dimension reduction, the important substructure 33e that contributes to the prediction accuracy is determined. Here, n′ is a natural number smaller than n, and is usually about 16 to 128.

The regression model construction unit 43f constructs the regression model 33f used in predicting the physical property value based on the important substructure 33e (in (g) of FIG. 5). The regression model 33f, whose dimensions are reduced to several tens dimensions, is obtained. The regression model 33f is expressed by the following equation (1) that obtains the predicted value y of the physical property of the polymer by the substructure number density vector x (i.e., the substructure number densities 33d).

y=f(x) (1)

When dimensions are not reduced, the number of substructure number density vector x is n, which is the number of dimensions. But after dimensions are reduced, the dimensions of the substructure number density vector x are n′. Because the predicted values are calculated using only the dimensions contributing to the prediction accuracy by using the important substructure 33e, the physical property prediction process can be efficiently performed.

The above-described process from (a) to (e) in FIG. 5 corresponds to the model generating process of the model generator 41. After the model generating process has been performed, in response to receiving the input of the polymer structure 6a desired to be processed from the user, the physical property prediction process is performed by the physical property prediction unit 45.

In response to the input of the polymer structure A of which the physical property is to be obtained, the physical property prediction unit 45 generates the substructure number density vector x_{A}, and predicts the physical property value y_{A} (in (h) of FIG. 5). The polymer structure input unit 47a inputs the polymer structure A, and the polymer physical property prediction unit 47b predicts the physical property value y_{A} by using the regression model 33f.

The physical property value y_{A} with respect to the polymer structure A desired to be processed can be obtained by substituting the substructure number density x_{A} in the above equation (1).

y_{A}=f(x_{A}) (2)

If the regression model 33f is for only one physical property, the value of a predetermined physical property is predicted. If the regression model 33f is obtained with respect to each of the various physical properties, the user may specify the polymer structure A and all physical properties or a specific physical property. In this case, the polymer physical property prediction unit 47b predicts the physical property value 6b by using the regression model 33f corresponding to each physical property and the important substructure 33e.

The process of calculating the numbers of substructures 33b in (c) of FIG. 5 and the process of calculating the number of atoms in (d) of FIG. 5 may be performed in any order. After calculating the number of atoms 33c is performed, the numbers of substructures 33b may be calculated, or the number of atoms 33c and the numbers of substructures 33b may be calculated in parallel. In the present embodiment, a “predicted value” of the physical property and a predicted “physical property value” are identical.

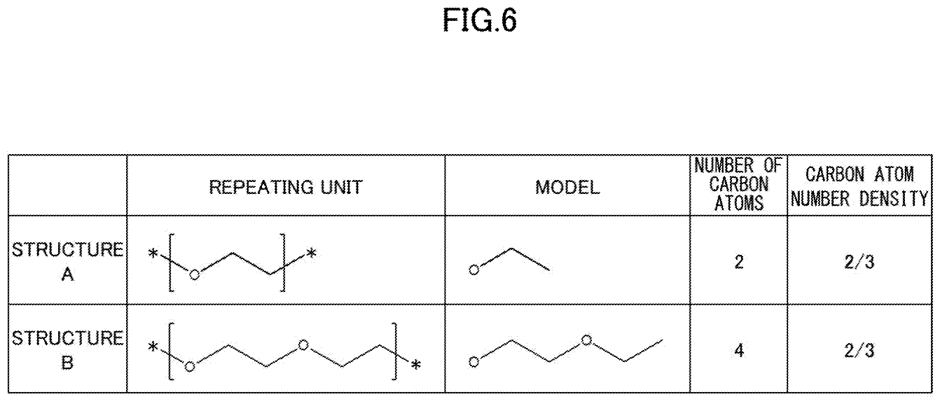

In the first functional configuration example, the substructure number density calculating unit 43d of the prediction processing unit 40 can recognize whether polymer structures are identical. FIG. 6 is a drawing illustrating an example of the number density. In FIG. 6, the number of carbons of a polyethylene oxide is illustrated as an example.

Referring to FIG. 6, when a structure A and a structure B are compared, a notation of the structure B is longer and different from a notation of the structure A. However, polymers represented by the structure A and the structure B are identical.

In an existing technique in which the number of substructures is used as a structure descriptor, the number of carbons of the structure A is “2,” the number of carbons of the structure B is “4”. Since the numbers of carbons of the structure A and the structure B are different, the polymers are mistakenly recognized as being different. This example indicates that when polymers having different lengths of the repeating unit are compared, the number of substructures cannot appropriately capture structural features of the polymer.

In the first functional configuration example, the substructure number density calculating unit 43d obtains the same substructure number density (carbon number density) “⅔” with respect to the structure A and the structure B. Thus, the dimension reduction unit 43e and the regression model construction unit 43f can correctly recognize that the structure A and the structure B represent the same polymer. Based on these examples, the inventors have found that by using the substructure number density 33d, polymers having different lengths of the repeating unit can be treated equivalently.

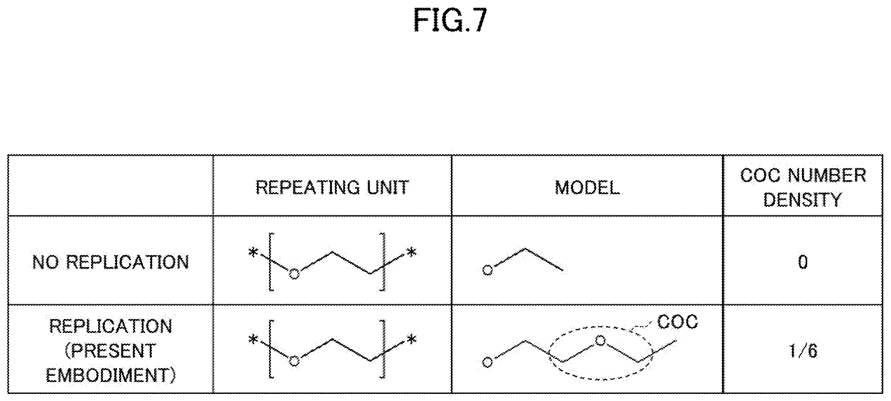

The inventors have also found that replicating and linking the repeating unit can improve the existing technique having low prediction accuracy. A difference between when replication is performed and when no replication is performed is described in FIG. 7. FIG. 7 is a drawing for describing the difference between when replication of the repeating unit is performed and when no replication is performed. In FIG. 7, a polyethylene oxide is used as an example. A repeating unit when replication is performed and a repeating unit when no replication is performed are identical.

When the repeating unit is not replicated, there is no substructure “COC”, which is expected to exist, at both ends of the repeating unit. Thus, COC number density is “0”. With respect to this, in the first functional configuration example, the repeating unit is replicated and the right side of the repeating unit is linked to an oxygen atom on the left side of the next repeating unit. Two repeating units causes the substructure “COC” to appear and “⅙” is obtained with respect to the number density of “COC”, which is expected to exist. In the first functional configuration example, the regression model 33f is obtained using the substructure number density 33d obtained in such a manner, thereby improving the prediction accuracy of the existing technique.

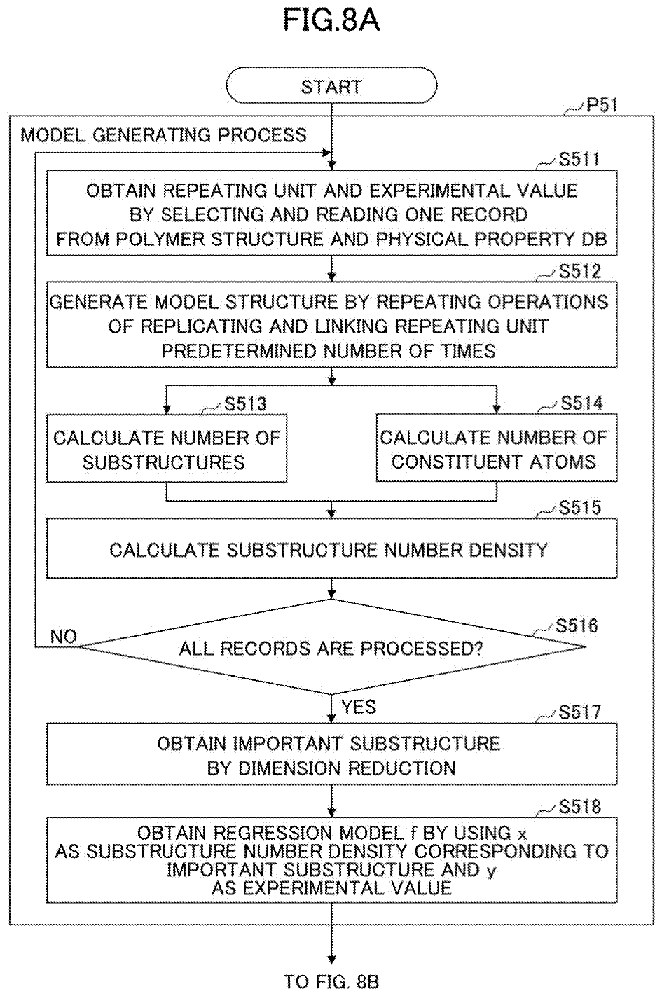

As described above, the prediction process is performed by the prediction processing unit 40 as illustrated in FIGS. 8A and 8B. FIGS. 8A and 8B are flowcharts for describing the prediction process. First, a model generating process P51 is performed by the model generator 41.

In step S511, in the model generator 41, the model structure generator 43a selects and reads one record from the polymer structure and physical property DB 31, and obtains the repeating unit and the experimental value. In step S512, the model structure generator 43a repeats an operation of replicating and linking the obtained repeating unit a predetermined number of times to generate the model structure 33a.

In step S513, the substructure number calculating unit 43b calculates the number of substructures 33b of the model structure 33a by using a predetermined structure descriptor (e.g., ECFP). In step S514, the atom calculating unit 43c calculates the number of atoms 33c of the model structure 33a. Step S513 may be performed after step S514 has been performed. Alternatively, steps S513 and S514 may be performed in parallel.

In step S515, the substructure number density calculating unit 43d calculates the substructure number density 33d by dividing the number of substructures 33b by the number of atoms 33c. As described above, the numbers of substructures 33b and the substructure number densities 33d are represented by an n-dimensional vector.

In step S516, the model generator 41 determines whether all records of the polymer structure and the physical property DB 31 have been completed. When all records have not been completed (NO in step S516), the model generator 41 returns to step S511 and performs a process similar to the process described above.

When all records have been completed (YES in step S516), the dimension reduction unit 43e performs a first regression analysis for obtaining a temporal regression model f′ by using the substructure number density 33d corresponding to the important substructure 33e as a variable x′ and an experimental value as y′. In step S517, the dimension reduction unit 43e reduces the dimensions of the temporal regression model f′ by a predetermined dimension reduction method and obtains the important substructure 33e.

Next, the regression model construction unit 43f performs a second regression analysis. In step S518, the regression model construction unit 43f obtains the regression model f by using the substructure number density 33d corresponding to the important substructure 33e as the variable x and the experimental value as y. The experimental value y is a value determined by the polymer name and the physical property in the polymer structure and physical property DB 31. The regression model f, that is, the regression model 33f, is stored in the storage unit 130.

When the regression model f is obtained for all or some of the physical properties registered in the polymer structure and physical property DB 31, an experimental value may be obtained for each physical property, and steps S517 to S518 may be repeated.

Subsequently, the physical property prediction process P55 is performed by the physical property prediction unit 45. When the polymer structure input unit 47a receives the polymer structure 6a in the prediction processing unit 40 (step S521), the model structure, the number of substructures, the number of atoms, and the substructure number density are obtained using the polymer structure 6a as the repeating unit (step S522). The model structure, the number of substructures, the number of atoms, and the substructure number density of the polymer structure 6a are obtained by performing a process similar to the process from step S512 to step S515 in the model generating process P51.

In step S524, the polymer physical property prediction unit 47b predicts the physical property value 6b based on the important substructure 33e obtained in step S517 by using the regression model f (i.e., the regression model 33f stored in the storage unit 130) obtained in step S518 by reducing the dimensions of the substructure number density of the polymer structure 6a. The obtained physical property value 6b is sent to the terminal 3 or displayed on the display device 15.

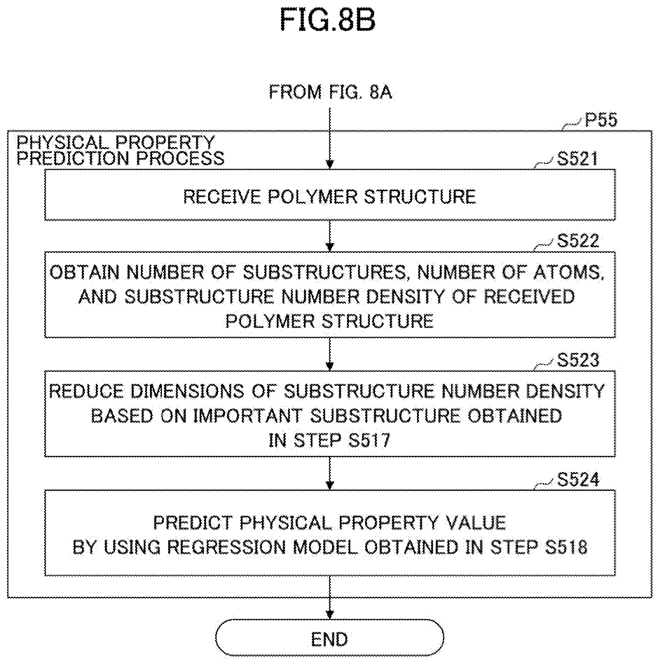

When the regression model 33f corresponding to each of multiple physical properties is generated, it is desirable to manage the generated regression models 33f in a table as illustrated in FIG. 9. FIG. 9 is a drawing illustrating an example of a data configuration of a regression model management table.

In FIG. 9, a regression model management table 33k is a table for managing the regression model 33f for each physical property, and includes items such as the physical property, the regression model, and the important substructure. The physical property represents a name for specifying the physical property, such as the refractive index, and the glass transition point. The regression model represents the regression model 33f corresponding to the physical property, constructed in the regression model construction unit 43f.

The important substructure specifies a valid vector of the vector of the substructure number density 33d, obtained after dimension reduction (i.e., a valid vector after dimension reduction) for using the regression model 33f. When the dimension reduction is omitted, the item of the important substructure is omitted.

In the physical property prediction unit 45, the polymer physical property prediction unit 47b obtains the regression model 33f and the important substructure 33e that are corresponding to the physical property to be predicted from the regression model management table 33k.

The inventors have verified the prediction accuracy of various functional configurations described below. According to verification results, the first functional configuration example described above can improve the prediction accuracy the most. Further, the inventors have verified that the prediction accuracy can be sufficiently improved even in a configuration in which either or both of the model structure generator 43a and the dimension reduction unit 43e are removed.

First, other examples of the functional configuration in which the prediction accuracy is verified will be described below. In the following description, a second functional configuration example is a functional configuration in which both of the model structure generator 43a and the dimension reduction unit 43e are removed, a third functional configuration example is a functional configuration in which the model structure generator 43a is removed, and a fourth functional configuration example is a functional configuration. in which the dimension reduction unit 43e is removed. In the second functional configuration example to the fourth functional configuration example, processing units similar to the processing units of the first functional configuration example illustrated in FIG. 3 are referenced by the same reference numerals, and the detailed description is omitted.

[Second Functional Configuration Example]

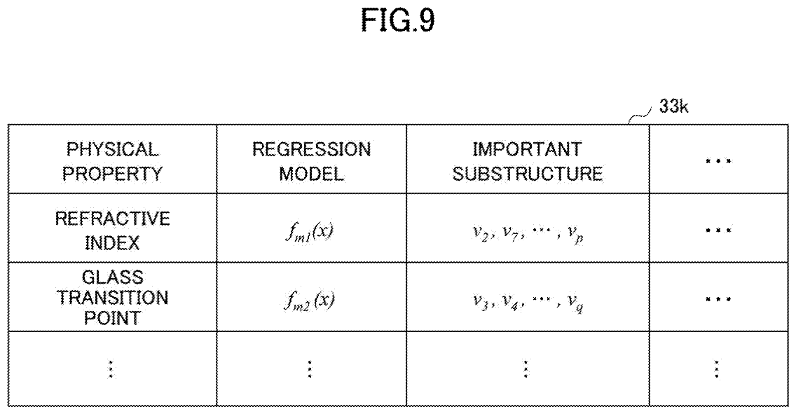

FIG. 10 is a drawing illustrating the second functional configuration example of the physical property prediction device. In FIG. 10, in the second functional configuration example, the model structure generator 43a and the dimension reduction unit 43e are removed from the first functional configuration example illustrated in FIG. 3. Thus, the model structure 33a and the important substructure 33e are not generated.

In the model generator 41, the substructure number calculating unit 43b and the atom calculating unit 43c obtains the number of substructures 33b and the number of atoms 33c by obtaining the repeating unit from the polymer structure and physical property DB 31.

Also in the second functional configuration example, the substructure number density calculating unit 43d calculates the substructure number density 33d. Then, the regression model construction unit 43f obtains the regression model 33f by using the calculated substructure number density 33d and the experimental value of the physical property.

In the physical property prediction unit 45, when the polymer structure input unit 47a inputs the polymer structure 6a, the polymer physical property prediction unit 47b calculates the number of substructures and the number of atoms by using the polymer structure 6a without creating the model structure 33a. The polymer physical property prediction unit 47b obtains the substructure number density by dividing the obtained number of substructures by the number of atoms, and uses the substructure number density 33d to obtain the property value 6b by the regression model 33f.

In the second functional configuration example, the regression analysis is performed only once. The number of dimensions of the regression model 33f is the same number as the number of dimensions of the substructure number densities 33d, and the important substructure 33e is not determined. However, if the values of the substructure number density 33d are identical, even polymers with different lengths of the repeating unit can be treated equivalently, and the prediction accuracy can be sufficiently improved.

[Third Function Configuration Example]

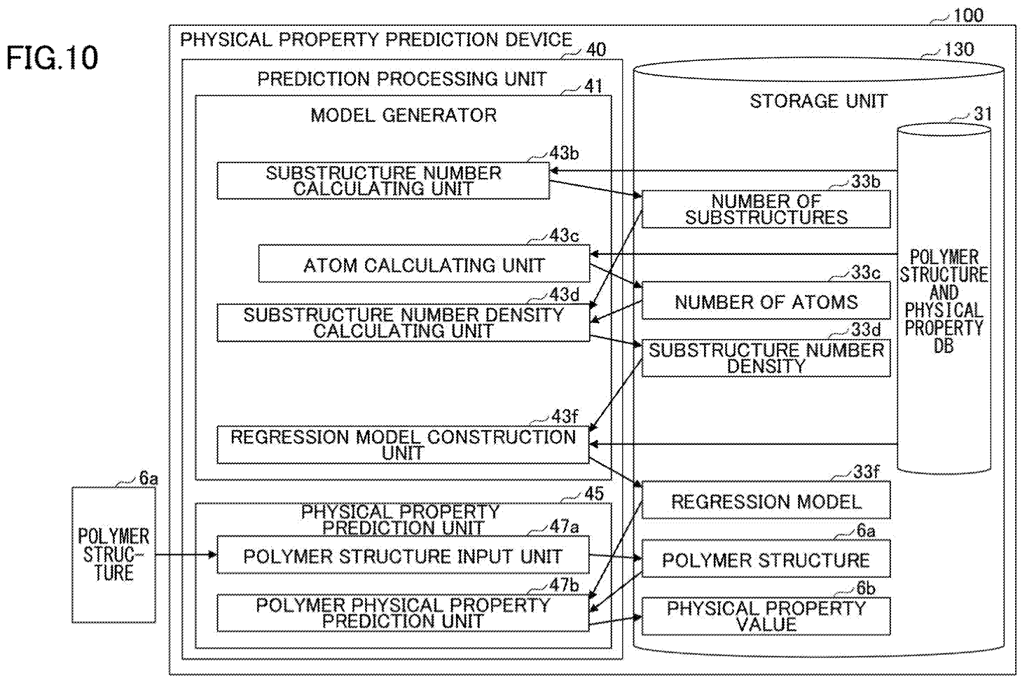

FIG. 11 is a drawing illustrating the third functional configuration example of the physical property prediction device. In FIG. 11, in the third functional configuration example, the model structure generator 43a is removed from the first functional configuration example illustrated in FIG. 3. Thus, the model structure 33a is not generated.

In a manner similar to the second functional configuration example, in the model generator 41, the substructure number calculating unit 43b and the atom calculating unit 43c obtain the repeating unit from the polymer structure and physical property DB 31 to obtain the number of substructures 33b and the number of atoms 33c. The substructure number density calculating unit 43d obtains the substructure number density 33d. Then, the dimension reduction unit 43e obtains the important substructure 33e and reduces the dimensions of the substructure number densities 33d, and the regression model construction unit 43f outputs the regression model 33f.

In the physical property prediction unit 45, when the polymer structure input unit 47a inputs the polymer structure 6a, the polymer physical property prediction unit 47b calculates the number of substructures and the number of atoms by using the polymer structure 6a without generating the model structure 33a. Then, the polymer physical, property prediction unit 47b obtains the substructure number density by dividing the obtained number of substructures by the number of atoms, reduces the number of dimensions of the substructure number densities based on the substructure number density 33d, and outputs the property value 6b by using the regression model 33f.

In the third functional configuration example, when the values of the substructure number density 33d are identical, even polymers with different lengths of the repeating unit can be equivalently treated. Although the model structure 33a is not generated, the important substructure 33e is determined by the dimension reduction unit 43e, thereby improving the prediction accuracy of the regression model 33f generated by the regression model construction unit 43f.

[Fourth Functional Configuration Example]

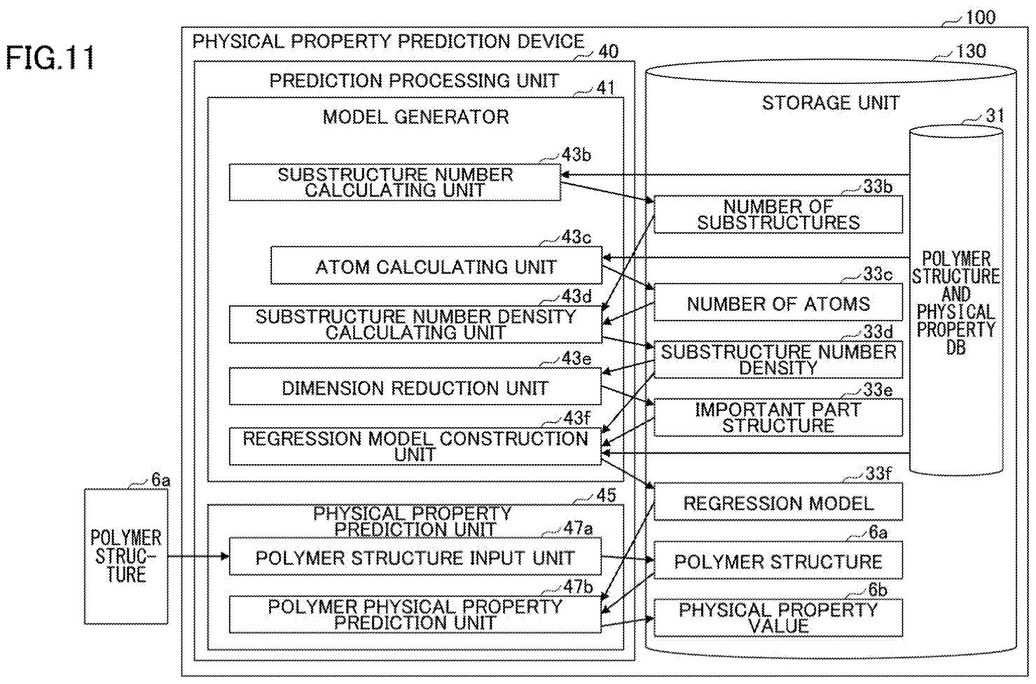

FIG. 12 is a drawing illustrating the fourth functional configuration example of the physical property prediction device. In FIG. 12, in the fourth functional configuration example, the dimension reduction unit 43e is removed from the first functional configuration example illustrated in FIG. 3. Thus, the important substructure 33e is not generated.

In the model generator 41, the model, structure generator 43a generates the model structure 33a, and the substructure number calculating unit 43b and the atom calculating unit 43c obtain the number of substructures 33b and the number of atoms 33c based on the model structure 33a.

In the physical property prediction unit 45, when the polymer structure input unit 47a inputs the polymer structure 6a, the polymer physical property prediction unit 47b generates the model structure 33a and calculates the number of substructures and the number of atoms by using the model structure 33a. Then, the polymer physical property prediction unit 47b obtains the substructure number density by dividing the obtained number of substructures by the number of atoms, and uses the substructure number density 33d to obtain the physical property value 6b by the regression model 33f. The dimensions of the substructure number densities obtained from polymer structure 6a are not reduced.

In the fourth functional configuration, when the values of the substructure number density 33d are identical, even polymers with different lengths of the repeating unit can be equivalently treated. Although the important substructure 33e is not generated, the model structure 33a and the substructure number density 33d can improve the prediction accuracy of the regression model 33f generated by the regression model construction unit 43f.

Next, a functional configuration example based on the existing technique will be described as a comparative example.

[Existing Functional Configuration Example]

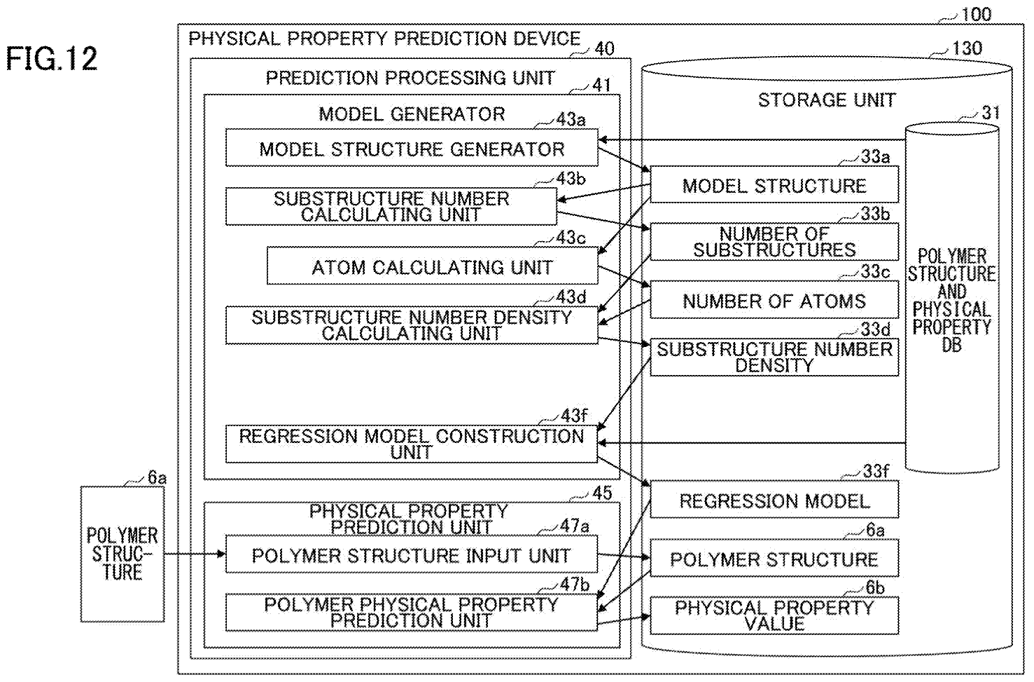

FIG. 13 is a drawing illustrating a functional configuration example of a physical property prediction device based on the existing technique. In FIG. 13, a physical property prediction device 9 has a functional configuration based on the existing technique. In the comparative example illustrated in FIG. 13, a prediction processing unit 90 of the physical property prediction device 9 includes a model generator 91 and a physical property prediction unit 95.

The model generator 91 is a processing unit that generates a regression model 83f and includes a substructure number calculating unit 93b and a regression model construction unit 93f. The substructure number calculating unit 93b obtains the repeating unit from the polymer structure and physical property DB 31 and stores the number of substructures 83b in the storage unit 130. The regression model construction unit 93f constructs the regression model 83f by performing a regression analysis using the number of substructures 83b and an experimental value obtained from the polymer structure and physical property DB 31, and stores the regression model 83f in the storage unit 130.

The physical property prediction unit 95 is a processing unit that predicts the physical property value by applying the polymer structure 6a desired to be processed by a user to the regression model 83f, and includes a polymer structure input unit 97a and a polymer physical property prediction unit 97b. The polymer structure input unit 97a stores the polymer structure 6a in the storage unit 130 when receiving the polymer structure 6a. The polymer physical property prediction unit 97b obtains the number of substructures of the polymer structure 6a and applies the obtained number of substructures to the regression model 83f to obtain the predicted physical property value 6b. The physical property value 6b is presented to the user.

[Verification of the Prediction Accuracy]

Next, results of verifying the prediction accuracy of the functional configuration example as a comparative example and the first to fourth functional configuration examples will be described. In each functional configuration example, the inventors have verified the prediction accuracy when the refractive index is predicted.

### EXAMPLE 1

In Example 1, the second functional configuration example (FIG. 10) in which the model structure generator 43a and the dimension reduction unit 43e are removed was used for the verification. In Example 1, the model structure 33a is not generated. In addition, the regression analysis is performed only once by the regression model construction unit 43f.

In the above-described second functional configuration example, structures of 51 general infinite linear polymers, such as polyethylene and polypropylene, (i.e., repeated units) and experimental values of the refractive index were used as training data. ECFP4 was used as the structure descriptor. Here, ECFP4 indicates ECFP having a radius of 4 and 1024 dimensions. Hereinafter, ECPF4 will be referred to as an ECFP descriptor.

The ECFP descriptor is represented by a vector, each dimension represents a substructure classification, and a value of each dimension corresponds the number of substructures. Software such as RDKit was used to generate the ECFP descriptor. For convenience, the ECFP Descriptor is defined as a substructure number descriptor. Then, a substructure number density descriptor is obtained by dividing each element of the substructure number descriptor by the number of constituent atoms.

As a regression method, the Gaussian process regression was used. The regression model 33f was constructed using the substructure number density descriptor as an explanatory variable and the experimental value of the refractive index as an objective variable.

A data set was divided into a training data set and a verification data set by leave-one-out cross-verification, a regression model was constructed after hyperparameter tuning was performed using the training data set, and the refractive index of a polymer that was not learned was predicted using the verification data set. The coefficient of determination R² and the root mean squared error (RMSE) were used as evaluation measures of the prediction accuracy. There are various definition equations of R², but the following equation was used this time.

$${R}^{2} ≡ 1 - \frac{{\sum }_{i} {( {y}_{i} - {f}_{i} )}^{2}}{{\sum }_{i} {( {y}_{i} - \overline{y} )}^{2}} \qquad [ Eq . 1 ]$$

Here, yᵢ is an experimental value of an ith polymer, y_bar is a measured average value, and fᵢ is the predicted value of the ith polymer.

In the description above, the number of constituent atoms corresponds to the number of atoms of the repeating unit, the substructure number descriptor corresponds to the numbers of substructures 33b, and the substructure number density descriptor corresponds to the substructure number densities 33 d.

### EXAMPLE 2

In Example 2, the third functional configuration example (FIG. 11) in which the model structure generator 43a was removed was used for the verification. Since the model structure 33a was not generated, the substructure number density 33d was obtained from the repeating unit of the polymer structure and physical property DB 31. The regression analysis was performed twice by the dimension reduction unit 43e and the regression model construction unit 43f.

Before constructing the regression model 33f, the dimension reduction unit 43e constructs a Lasso regression model (corresponding to the temporal regression model) from the substructure number density 33d and the experimental value of the physical property of the polymer obtained from the polymer structure and physical property DB 31, and performs the dimension reduction by excluding variables with coefficients of 0. In the regression model construction unit 43f, the regression model 33f was constructed by the Gaussian process regression in a manner similar to Example 1. Then, the prediction accuracy of the refractive index was verified by a method similar to the method of Example 1.

### EXAMPLE 3

In Example 3, the fourth functional configuration example (FIG. 12) in which the dimension reduction unit 43e is removed was used for the verification. The important substructure 33e was not obtained. The regression model 33f with the number of dimensions of the substructures number densities 33d was constructed.

First, after 10 repeating units were generated as the model structure 33a, the substructure number descriptor and the number of atoms were calculated, and the substructure number density descriptor was calculated. After the substructure number density descriptor was calculated, a predicted value of the refractive index was obtained by a method similar to the method of Example 1, and the prediction accuracy was verified.

### EXAMPLE 4

In Example 4, the first functional configuration example (FIG. 3) including the important substructure 33e and the dimension reduction unit 43e was used for the verification.

First, after 10 repeating units were. generated as the model structure 33a, the substructure number descriptor and the number of atoms were calculated, and the substructure number density descriptor was calculated. In addition, the dimension reduction unit 43e constructed a Lasso regression model (corresponding to the temporal regression model) and constructed the regression model 33f by the Gaussian process regression after reducing the dimensions by excluding variables with coefficients of 0. Then, the prediction accuracy of the refractive index was verified by a method similar to the method of Example 1.

### COMPARATIVE EXAMPLE 1

In Comparative example 1, the existing functional configuration example (FIG. 13) in which only the substructure number calculating unit 93b and the regression model construction unit 93f obtains the regression model 83f was used for the verification.

The substructure number calculating unit 93b obtained the regression model 83f from the number of substructures 83b (i.e., the substructure number descriptor) obtained using the repeating unit and the experimental value of the refractive index. The regression model 83f was constructed using the Gaussian process regression in a manner similar to the manner of Example 1. The predictive accuracy was verified in a manner similar to the manner of Example 1.

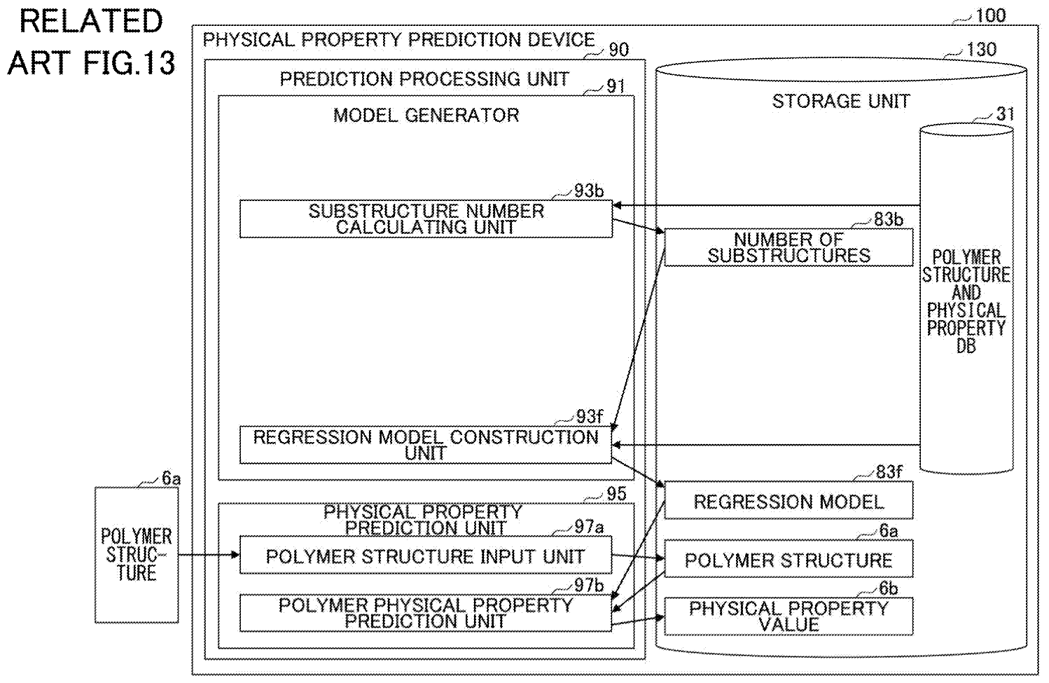

FIG. 14 is a drawing illustrating an example of evaluation results. FIG. 14 illustrates the evaluation results of the prediction accuracy of the refractive index evaluated for each of Comparative example 1 and Examples 1 to 4 as described above. Results of R² and RMSE are shown as the evaluation results.

In the evaluation result of R², compared with “−0.063” of Comparative Example 1, results of “0.890” in Example 1, “0.900” in Example 2, “0.932” in Example 3, and “0.950” in Example 4 are shown. The closer to “1” R² is, the higher the prediction accuracy becomes. Thus, from the evaluation results, it is found that the prediction accuracy can be significantly improved in any of the functional configuration examples according to the present embodiment.

In RMSE, compared with “0.084” in Comparative example 1, results of “0.027” in Example 1, “0.026” in Example 2, “0.021” in Example 3, and “0.018” in Example 4 are shown. The closer to “0” RMSE is, the higher the prediction accuracy becomes. Thus, it is found that the prediction accuracy can be significantly improved in all the functional configuration examples according to the present embodiment in the evaluation results.

From such evaluation results, it can be said that the substructure number density 33d, which the inventors have focused on, greatly contributes to the improvement of the prediction accuracy in the present embodiment.

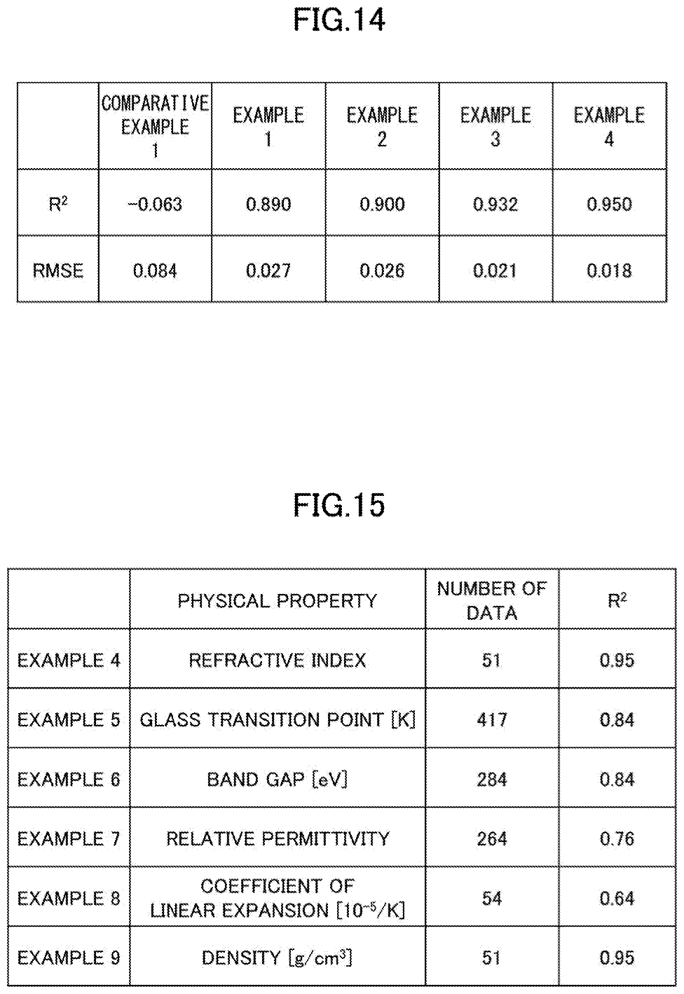

FIG. 15 illustrates an example of results of an evaluation performed by changing the physical property and the number of data in the data set in a manner similar to the manner of Example 4. FIG. 15 illustrates evaluation results of physical properties of Examples 5 to 9 performed by a respective number of data in a manner similar to the manner of Example 4. Results of R² are shown as the evaluation results.

In the results of R², results of a refractive index, a glass transition point [k], a band gap [eV], a relative permittivity, a coefficient of linear expansion [10⁻⁵/K], and a density [g/cm³] are greater than or equal to 0.6. Thus, it is found that the prediction accuracy can be significantly improved.

Since the physical property value can be predicted in advance using the physical property prediction device 100, the polymer structure 6a to be manufactured can be accurately selected. Thus, it is possible to reduce waste of costs, time, and so on related to polymer manufacturing.

As described above, according to the present embodiment, the physical properties of the polymer can be accurately predicted by generating the structure descriptor based on the polymer structure.

Next, a manufacturing method of a polymer using the desired polymer structure 6a and the predicted physical property will be described. FIG. is a drawing illustrating an example of a manufacturing method of the polymer.

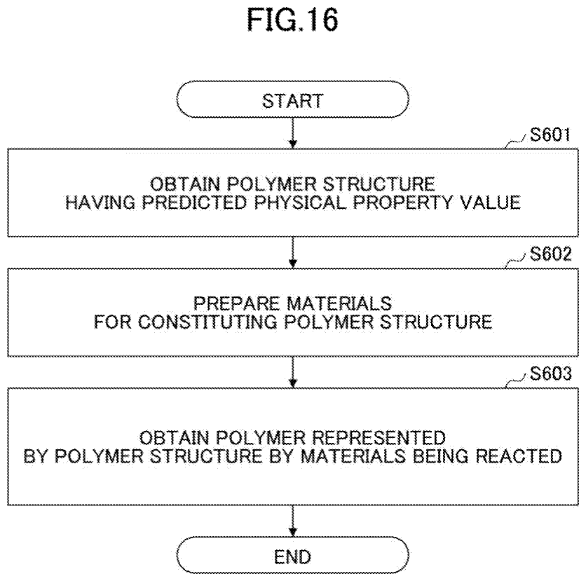

From FIG. 16, the manufacturing method of the polymer in the present embodiment includes a step of obtaining a polymer structure having a property value predicted by the polymer physical property prediction method according to the present embodiment (S601) and a step of preparing raw materials for forming the polymer structure (S602), and a step of causing the raw materials to react to obtain a polymer represented by the polymer structure (S603).

In step S601, a polymer structure predicted to have a predetermined physical property value is appropriately selected from the input polymer structures. For example, a polymer structure whose refractive index is within a predetermined range may be selected from the input polymer structures.

In step S602, raw materials of monomers, oligomers, and the like are prepared as raw materials for forming the polymer structure selected in step S601. For example, if the selected polymer structure is polyethylene, ethylene is prepared.

In step S603, the raw materials prepared in S602 are caused to react to obtain a polymer represented by the polymer structure selected in S601. For example, ethylene is polymerized to form polyethylene.

The present invention is not limited to the specifically disclosed embodiments and various variations and modifications can be made without departing from the scope of the claims.

The present application is based on and claims priority to Japanese Patent Application No. 2018-043171, filed on Mar. 9, 2018, the entire contents of which are hereby incorporated herein by reference.

What is claimed is:

1. A polymer physical property prediction device comprising:
a processor; and
a memory storing program instructions that cause the processor to
read a structural unit from a storage unit and use the structural unit to calculate numbers each indicating how many substructures are in a polymer, the polymer being formed of repetition of the structural unit;
calculate a number indicating how many atoms are in the structural unit;
calculate number densities of the substructures from the numbers of substructures and the number of atoms in the structural unit;
construct a regression model that predicts a physical property value by using an experimental value of a physical property of the polymer and the number densities of the substructures, the experimental value being obtained from the storage unit;
input a polymer structure of which the physical property value is to be predicted; and
predict the physical property value corresponding to the input polymer structure by using the regression model.

2. The polymer physical property prediction device as claimed in claim 1, wherein the program instructions cause the processor to further classify the substructures of the polymer using an algorithm, and the calculated numbers of substructures each indicate a number of each corresponding classified substructure present in the polymer.

3. The polymer physical property prediction device as claimed in claim 1, wherein the program instructions cause the processor to further remove part of the calculated number densities of the substructures.

4. The polymer physical property prediction device as claimed in claim 1, wherein the program instructions cause the processor to further generate a model structure by repeatedly using the structural unit obtained from the storage unit.

5. The polymer physical property prediction device as claimed in claim 1,
wherein the program instructions cause the processor to further
generate a model structure by repeatedly using the structural unit obtained from the storage unit, and
remove part of a structure descriptor representing the calculated number densities of the substructures,
wherein the numbers of substructures and the number of atoms are calculated from the generated model structure.

6. The polymer physical property prediction device as claimed in claim 1, wherein the program instructions cause the processor to calculate the numbers of substructures and the number of atoms in the polymer structure by using the input polymer structure, calculate the number densities of the substructures from the calculated numbers of substructures and the calculated number of atoms, and apply the calculated number densities to the constructed regression model to predict the physical property value of the polymer structure.

7. The polymer physical property prediction device as claimed in claim 3, wherein the program instructions cause the processor to calculate the numbers of substructures and the number of atoms of the polymer structure by using the input polymer structure, calculate the number densities of the substructures from the calculated numbers of substructures and the calculated number of atoms, remove part of the calculated number densities of the substructures, and apply the number densities whose part is removed to the constructed regression model to predict the physical property value of the polymer structure.

8. The polymer physical property prediction device as claimed in claim 4, wherein the program instructions cause the processor to generate the model structure by repeatedly using the input polymer structure, calculate the numbers of substructures and the number of atoms in the model structure, calculate the number densities of the substructures from the calculated numbers of substructures and the calculated number of atoms, and apply the calculated number densities to the constructed regression model to predict the physical property value of the polymer structure.

9. The polymer physical property prediction device as claimed in claim 5, wherein the program instructions cause the processor to generate the model structure by repeatedly using the input polymer structure, calculate the numbers of substructures and the number of atoms in the model structure, calculate the number densities of the substructures from the calculated numbers of substructures and the calculated number of atoms, remove the part of the structure descriptor representing the calculated number densities of the substructures, and apply the number densities whose part is removed to the constructed regression model to predict the physical property value of the polymer structure.

10. A non-transitory computer-readable recording medium having stored therein a polymer physical property prediction program for causing a computer to execute a process comprising:
reading a structural unit from a storage unit and using the structural unit to calculate numbers each indicating how many substructures are in a polymer, the polymer being formed of repetition of the structural unit;
calculating a number indicating how many atoms are in the structural unit;
calculating number densities of the substructures from the numbers of substructures and the number of atoms in the structural unit;
constructing a regression model that predicts a physical property value by using an experimental value of a physical property of the polymer and the number densities of the substructures, the experimental value being obtained from the storage unit;
inputting a polymer structure of which the physical property value is to be predicted; and
predicting the physical property value corresponding to the input polymer structure by using the regression model.

11. The recording medium as claimed in claim 10, wherein the predicting the physical property value further includes calculating the numbers of substructures and the number of atoms in the polymer structure by using the input polymer structure, calculating the number densities of the substructures from the calculated numbers of substructures and the calculated number of atoms, and applying the calculated number densities to the regression model to predict the physical property value of the polymer structure.

12. A polymer physical property prediction method executed by a computer, the method comprising:
reading a structural unit from a storage unit and using the structural unit to calculate numbers each indicating how many substructures are in a polymer, the polymer being formed of repetition of the structural unit;
calculating a number indicating how many atoms are in the structural unit;
calculating number densities of the substructures from the numbers of substructures and the number of atoms in the structural unit;
constructing a regression model that predicts a physical property value by using an experimental value of a physical property of the polymer and the number densities of the substructures, the experimental value being obtained from the storage unit;
inputting a polymer structure of which the physical property value is to be predicted; and
predicting the physical property value corresponding to the input polymer structure by using the regression model.

13. The polymer physical property prediction method as claimed in claim 12, wherein the predicting the physical property value further includes calculating the numbers of substructures and the number of atoms in the polymer structure by using the input polymer structure, calculating the number densities of the substructures from the calculated numbers of substructures and the calculated number of atoms, and applying the calculated number densities to the regression model to predict the physical property value of the polymer structure.

14. A polymer manufacturing method comprising:
obtaining a polymer structure having a physical property predicted by the polymer physical property prediction method as claimed in claim 12;
preparing materials for forming the polymer structure; and
obtaining a polymer represented by the polymer structure by reacting the materials.

15. The polymer physical property prediction device as claimed in claim 1, wherein the physical property value is a value of a refractive index or a value of a glass transition point.

16. The polymer physical property prediction device as claimed in claim 1, wherein the physical property value is a value of a band gap, a value of a relative permittivity, a value of a coefficient of linear expansion, or a value of a density.
